(19) United States
(12) Patent Application Publication
(10) Pub. No.: US 2001/0031474 A1
(43) Pub. Date: Oct. 18, 2001

(54) Chimeric neuropeptide Y receptors

(75) Inventors: Michele Bennett Kinrade, Northford, CT (US); Robbin M. Brodbeck, Madison, CT (US); James E. Krause, Madison, CT (US)

Correspondence Address: Ladas & Parry, 26 West 61st Street, New York, NY, 10023, US

(73) Assignee: NEUROGEN CORPORATION

(21) Appl. No.: 09/771,956

(22) Filed: Jan. 29, 2001

Related U.S. Application Data
(60) Provisional application No. 60/178,652, filed on Jan. 28, 2000.

(51) Int. Cl. G01N 33/53; C07H 21/04; C12N 5/06; C07K 14/705

(52) U.S. Cl. 435/7.1; 435/325; 530/350; 536/23.5

(57) ABSTRACT

Novel chimeric G-protein coupled receptors are provided as isolated polypeptides, membrane preparations containing such chimeric receptors, nucleic acids encoding such chimeric receptors, and cells expressing such receptors. The chimeric receptors are NPY5 receptors with most or all of either one or both of the third cytoplasmic loop domain or the C-terminal intracellular domain of NPY5 replaced with the corresponding region(s) of another NPY receptor, preferably an NPY1 receptor.

### BACKGROUND OF THE INVENTION

G Protein-Coupled Receptors (GPC's):

GPC's are a class membrane-spanning proteins that act to transude signals into the cell in response to stimulation by hormones, neurotransmitters, and other extracellular signaling molecules, including peptides and smaller organic molecules. See, e.g., Gather, et al., J. Biol. Chem., 273:17979-82, and 1998. Receptor polypeptides such as GPC's are typically found at very low concentrations on the cell surface. Because of their key roles in mediating cellular responses, GPC's are highly effective targets for drug action. Isolated GPC's, particularly as components of isolated membrane preparations, as well as cloned GPCR genes (preferably candies) and cells expressing such genes, are used in the pharmaceutical industry as the basis of drug discovery and development assays. Means to obtain artificially high concentrations of GPC's in cells and membranes are much sought after, as high levels of active receptors facilitate assays with higher sensitivity.

GPC's consist of a single contiguous amino acid chain comprising seven hydrophobic domains interconnecting eight hydrophilic domains. Once the amino acid sequence of a GPCR is determined, the precise locations of these domains may be conveniently calculated by computer analysis of hydrophobicity or hydrophilicity using hydropathy profiles, such as standard Kyte-Doolittle analysis (Kyte and Doolittle, J. Mol. Biol. 157:105-32, 1982). The transition boundaries between the hydrophobic and hydrophilic domains are typically marked by the presence of charged or polar (hydrophilic) amino acid residues at the beginning or end of a stretch of uncharged and nonpolar (hydrophobic) residues. The N-terminus of a cell surface GPCR extends into the extracellular space and the C-terminus into the cytoplasm of the cell. Each of the seven hydrophobic domains is about 20-25 amino acids long, assumes a largely alpha helical conformation, and crosses once through the plasma membrane, its entire extent generally embedded in the membrane. The hydrophobic domains of GPCRs are thus also referred to as transmembrane (TM) domains, membrane-spanning alpha helical domains, or the like, while the hydrophilic domains are referred to as either extracellular or intracellular domains, depending upon their predicted locations in a functional, membrane-bound GPCR. The hydrophilic domains interconnecting TM domains form loops within the cytoplasm or extracellular space, and are consequently referred to as cytoplasmic or extracellular loop domains.

GPCRs have been structurally modeled as to secondary and tertiary structural conformation, and the precise locations of the extracellular, TM and intracellular domains within their primary structures (i.e., their amino acid sequences) are well known and generally agreed to in the art (see, e.g., Baldwin, EMBO J. 12:1693-703, 1993, also see http://swift.embl-heidelberg.de/7tm/seq/snakes.html). These receptor proteins thus comprise an extracellular N-terminal domain, seven membrane-spanning alpha helical domains (connected by three intracellular loop domains alternating with three extracellular loop domains), and an intracellular C-terminal domain.

The locations of the various domains of neuropeptide Y (NPY) receptors can be readily determined by inspections of the “Viseur's snake like view” for the particular receptor polypeptide generated by the European Molecular Biology Laboratory's Viseur software. These Viseur's snake like views are electronically published for a wide variety of GPCR polypeptides (including NPY receptors of various mammalian and non-mammalian vertebrate species —http://swift.embl-heidelberg.de/7tm/seq/snakes.html). In these snake like views, the amino acids of the polypeptide sequence of the receptors are set forth as one-letter-code-containing circles. The TM domains are depicted as diagonally stacked circles to represent the alpha helical conformation believed to be adopted by of these domains in situ, while the other domains are depicted as vertically and horizontally arrayed sequences.

The precise structural characteristics (importantly including and largely flowing from the primary structure) of the extracellular and membrane spanning domains are believed to largely determine the ligand specificity of the receptor. In particular, peptide binding typically involves amino acid residues near the top of a plurality of the seven TM domains (i.e., TM domain residues adjacent to, generally within about ten to fifteen amino acids from, the extracellular domains) and within the extracellular domains of the receptors, while non-peptide type ligands are believed to typically bind deeper in the plane of the membrane, between several of the TM domains.

The precise structures of its third intracellular loop and intracellular C-terminal domain are believed to dictate important functional characteristics of GPCRs. In particular, they are believed to significantly contribute to the determination of the characteristics of the specific G-protein binding interactions of any particular GPCR, including any of the various neuropeptide Y (NPY) receptors, with any of the many subtypes of heterotrimeric G-proteins. As these subtypes are often functionally distinct, these changes in binding interactions are believed to result in alterations in receptor function. These domain structures are, of course, a function of the amino acid (primary) sequence of each domain.

Without wishing to be bound by any particular theory of operation, it is believed that these specific binding interactions are involved in bringing the G-protein into close proximity with the receptor's other intracellular domains (the three intracellular loops connecting six of the seven TM alpha helices), an action that is believed to be fundamental to determining the receptor's signal transduction functionality. Both the third intracellular loop and the C-terminal domain thus play key roles in determining the type of intracellular signal that is transmitted by a GPCR upon activation.

Signal transduction is initiated by the binding of an agonist ligand to the receptor. This elicits conformational changes in the extracellular domains. When the receptor is functioning properly, these conformational changes propagate through the TM domains and result in a coordinated change in the intracellular portions of the GPCR. This precise alteration in the intracellular domains acts to trigger the associated G-protein complex to modulate intracellular signaling. In particular, in an NPY receptor, the alteration triggers a GTP for GDP exchange on the G alpha subunit of the complex, the release of the G-protein complex from the receptor, and the dissociation of the G alpha from the G beta and G gamma subunits of the complex. The ultimate result of these alterations is the activation or inhibition of intracellular signaling systems.

Chimeric GPCRs:

In the course of analyzing the specific contributions of the various GPCR domains to receptor function, many different chimeric GPCR molecules with heterologous N-terminal and C-terminal domains have been constructed using recombinant DNA techniques. These efforts have yielded unpredictable results, depending upon the sources of the various domains being combined in a chimeric receptor. See, e.g., Blount, et al., J. Biol. Chem., 268:16388-95, 1993; Liggett, et al., Proc. Natl. Acad. Sci. USA, 90: 3665-69, 1993.

In some cases, attempts to express chimeric GPCR-encoding cDNAs (comprising certain combinations of DNA fragments encoding heterologous domains) result in a receptor that is poorly expressed at the cell surface. In other cases, the expressed chimeric receptors localize into different membranes than do native receptors. See, e.g., Moyle, et al., J. Biol. Chem., 266:10807-12, 1991; and Mery and Boulay, J. Biol. Chem., 269:3457-63, 1994.

Sometimes, in spite of proper membrane insertion, the combined heterologous domains do not function properly. Often the conformational changes in the extracellular domains triggered by the binding of an agonist ligand is not adequately propagated to the intracellular portions of the receptor, and thus fails to trigger the activation of the associated G protein to generate a sufficient modulation of intracellular signaling. Chimeric receptors may also exhibit altered ligand-binding specificity as compared to the native receptor from which the ligand-binding portion of the chimeric receptor has been obtained. See, e.g., Blount, et al., J. Biol. Chem., 268:16388-95, 1993; and Buggy, et al., J. Biol. Chem., 270:7474-78, 1995.

Native GPCRs transduce a cell surface agonist-binding event into an intracellular signal via the intervening actions of cytosolic heterotrimeric G-protein complexes. There is a growing list of heterotrimeric G-protein combinations demonstrated to couple to GPCRs. The G-protein complexes in turn activate specific effector proteins that continue the signal transduction process, typically by generating a second messenger such as cAMP, cGMP, inositol 1,4,5-bisphosphate or arachidonic acid. In normal GPCR function, a specific G-protein alpha beta and gamma subunit combination typically activates a specific effector protein, although some GPCRs have been shown to couple to multiple signal transduction pathways.

Assays of GPCR Function:

Assays allowing for the sensitive and accurate determination of GPCR function are much sought after, as they are useful research tools, e.g., for analyzing the effects of compounds that modulate GPCR function and thereby can act as drugs. For example, agonist-induced GTPγ³⁵S binding by GPCRs provides a functional measure of G-protein activation. Although some receptors may not provide optimal results in such assays, this type of assay has been widely used for many GPCRs. It is used, e.g., to distinguish agonists from antagonists and to determine the potency and efficacy of agonists for a given GPCR (see, e.g., Thomas et al., J. Recept Signal Transduct Res 15:199-211, 1995).

Robust functional activity assays are as yet available to measure only a limited subset of G-protein-mediated signaling pathways. Robust assays are those that can consistently provide signal-to-noise characteristics allowing for the acquisition of statistically significant data sets from quadruplicate, more preferably triplicate, and most preferably from duplicate sample runs. In all GPCR research, and particularly in the area of drug discovery, such robust assays facilitate the acquisition of useful and informative data.

The robustness of such an assay is dramatically influenced by the particular receptor used in the assay. Thus, GPCRs with signaling characteristics adapted so as to facilitate robust functional activity assays are particularly valuable research tools.

NPY and NPY Receptors:

Neuropeptide Y (NPY) consists of 36 amino acids and is one of the most abundant peptides present in the mammalian central and peripheral nervous systems. NPY exhibits a variety of potent central and peripheral effects including modulation of feeding, memory, blood pressure, cardiac contractility, and intestinal secretion. Classical pharmacological evidence suggests that NPY effects are mediated by a number of different GPCR subtypes. Y1, Y2, Y4, Y5, Y6 and Y7 receptors (alternatively referred to as NPY1, NPY2, NPY4, NPY5, NPY6, and NPY7 receptors) have all been cloned and recombinantly expressed. All known NPY receptors are G-protein-coupled transmembrane proteins with seven membrane spanning TM domains.

The best characterized of the NPY receptors is Y1, which has been cloned from the mouse (Eva, et al., FEBS Lett. 314:285, 1992), rat (Eva, et al., FEBS Lett. 271:80, 1990), and human (Larhammar, et al., J. Biol. Chem. 267:10935, 1992). It is considered to be postsynaptic and to mediate most of the peripheral actions of NPY, including vasoconstriction and increased arterial blood pressure (Larhammar, et al., J. Biol. Chem. 267:10935, 1992; Westfall, et al., Ann. NY Acad. Sci. 611:145, 1990). The Y1 receptor in the central nervous system has been associated with various effects of NPY, including its anxiolytic action, its effects on feeding behavior, and its reduction of spontaneous locomotor activity (see, e.g., Wahlestedt, et al., Science 259:528, 1993).

The NPY5 receptor has been suggested to play a key role in the modulation of feeding behavior. Studies of seizure-prone mice have led to the suggestion that the Y5 receptor may also have an anti-epileptic activity in the control of limbic seizures. Y5-like receptors have also been implicated in attenuation of morphine withdrawal symptoms, enhancement of diuresis and natriuresis, lowering of blood glucose, inhibition of luteinizing hormone secretion, and reduction of acetylcholine release in the ileum. See, for example, Hu, et al., J. Biol. Chem., 271:26315-19, 1996; Gerald, et al., Nature, 382:168-71, 1996; Blomqvist, et al., TINS, 20: 294-98, 1997. The sequences of Y1 and Y5 receptors of humans, dogs, mice, guinea pigs, rats, and Y1 receptors of sheep have all been reported and have been published, e.g., by Genbank (http://www.ncbi.nlm.nih.gov/).

Y1 receptors are structurally characterized as having a single polypeptide chain comprising, in N-terminal to C-terminal order, an NPY1 N-terminal extracellular domain, an NPY1 first TM domain, an NPY1 first intracellular loop domain, an NPY1 second TM domain, an NPY1 first extracellular loop domain, an NPY1 third TM domain, an NPY1 second intracellular loop domain, an NPY1 fourth TM domain, an NPY1 second extracellular loop domain, an NPY1 fifth TM domain, an NPY1 third intracellular loop domain, an NPY1 sixth TM domain, an NPY1 third extracellular loop domain, an NPY1 seventh TM domain, and an NPY1 C-terminal intracellular domain.

Y5 receptors are structurally characterized as having a single polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain, an NPY5 third intracellular loop domain, an NPY5 sixth TM domain, an NPY5 third extracellular loop domain, an NPY5 seventh TM domain, and an NPY5 C-terminal intracellular domain.

In the human Y1 receptor (DNA sequence—SEQ ID NO:1, amino acid sequence—SEQ ID NO:2), the third intracellular loop domain consists essentially of amino acids 232 (Phe) to 263 (Ile) of SEQ ID NO:2, as indicated, for example, by the Viseur's snake like view for this receptor (see, e.g., http://swift.embl-heidelberg.de/7tm/seq/vis/NY1R_HUMAN/NY1R_HUMAN.html). In accordance with the amino acid sequence residue charge/polarity considerations discussed above, the termini of this loop are preferably defined by the presence (within the domain) of a charged residue (Lys 233 of SEQ ID NO:2) located at the end of the long stretch of hydrophobic residues (the fifth TM domain) and a charged residue (Arg 260 of SEQ ID NO:2) located at the beginning of the long stretch of hydrophobic residues (the sixth TM domain).

In the rat Y1 receptor, the third intracellular loop domain consists essentially of amino acids 231 (Phe) to 262 (Val) of SEQ ID NO:3, as indicated, for example, by the Viseur's snake like view for this receptor (see, e.g., http://swift.embl-heidelberg. de/7tm/seq/vis/NY1R_RAT/NY1R_RAT.html). In accordance with the amino acid sequence residue charge/polarity considerations discussed above, the termini of this loop domain are preferably defined by the presence (within the domain) of a charged residue (Lys 232 of SEQ ID NO:3) located at the end of the long stretch of hydrophobic residues (the fifth TM domain) and another charged residue (Arg 259 of SEQ ID NO:3) located at the beginning of the long stretch of hydrophobic residues (the sixth TM domain).

The following discussion of human NPY5 domains illustrates the domain structure information available electronically for this receptor (see, e.g., http://swift.emblheidelberg.de/7tm/seq/vis/NY5R_HUMAN/NY5R_HUMAN.html).

In accordance with this information: A preferred Y5 N-terminal extracellular domain consists essentially of residues 1 (Met) to 50 (Leu) of SEQ ID NO:13.A preferred Y5 first TM domain consists essentially of residues 51 (Gln) to 71 (Leu) of SEQ ID NO:13. A preferred Y5 first intracellular loop domain consists essentially of residues 72 (Ile) to 84 (Thr) of SEQ ID NO: 13. A preferred Y5 second TM domain consists essentially of residues 85 (Thr) to 105 (Ser) of SEQ ID NO:13. A preferred Y5 first extracellular loop domain consists essentially of residues 106 (Pro) to 125 (His) of SEQ ID NO:13. A preferred Y5 third TM domain consists essentially of residues 126 (Ile) to 146 (Ala) of SEQ ID NO:13. A preferred Y5 second intracellular loop domain consists essentially of residues 147 (Ile) to 167 (Tyr) of SEQ ID NO:13. A preferred Y5 fourth TM domain consists essentially of residues 168 (Phe) to 188 (His) of SEQ ID NO:13. A preferred Y5 second extracellular loop domain consists essentially of residues 188 (Ser) to 220 (Ala) of SEQ ID NO:13. A preferred Y5 fifth TM domain consists essentially of residues 221 (Phe) to 241 (His) of SEQ ID NO:13. A preferred Y5 third intracellular loop domain consists essentially of residues 242 (Thr) to 378 (Tyr) of SEQ ID NO:13. A preferred Y5 sixth TM domain consists essentially of residues 379 (Arg) to 401 (Thr) of SEQ ID NO:13. A preferred Y5 third extracellular loop domain consists essentially of residues 402 (Arg) to 414 (Lys) of SEQ ID NO:13. A preferred Y5 seventh TM domain consists essentially of residues 415 (Leu) to 438 (Leu) of SEQ ID NO:13. A preferred Y5 C-terminal intracellular domain consists essentially of residues 439 (Asn) to 455 (Met) of SEQ ID NO:13.

The following discussion of human NPY1 domains illustrates the domain structure information available electronically for this receptor (see, e.g., http://swift.embl-heidelberg.de/7tm/seq/vis/NY1R_HUMAN/NY1R_HUMAN.html). This Viseur's snake like view also indicates numerous points at which variant sequences for human NPY1 have been found or have been created.

In accordance with this information: A preferred Y1 fifth TM domain consists essentially of residues 211 (Tyr) to 231 (Tyr) of SEQ ID NO:2. A preferred Y1 third intracellular loop domain consists essentially of residues 232 (Phe) to 263 (Ile) of SEQ ID NO:2. A preferred Y1 sixth TM domain consists essentially of residues 264 (Met) to 286 (Phe) of SEQ ID NO:2. A preferred Y1 seventh TM domain consists essentially of residues 300 (Leu) to 323 (Leu) of SEQ ID NO:2. A preferred Y1 C-terminal intracellular domain consists essentially of residues 324 (Asn) to 384 (Ile) of SEQ ID NO:2.

Further discussions of NPY, GPCR, and NPY-receptor structure, physiology, and pharmacology (including NPY-receptor and other GPCR domain structure and nomenclature) are presented in U.S. Pat. No. 6,001,970, issued on Dec. 14, 1999 in the names of Margaret A. Cascieri, Douglas John MacNeil, and Catherine D. Strader, which is incorporated herein by reference for its teachings in such regard at columns 1-5, 6 (lines 1-12, 30-45, and 64-67) 7-8, and 9 (lines 1-35) and FIGS. 1-3.

Further discussions of Y1 and Y5 receptors are presented in US Pat. No. 5,571,695 issued Nov. 5, 1996, in the names of Lisa Selbie, Herbert Herzog, and John Shine; U.S. Pat. No. 5,965,392, issued Oct. 12, 1999, in the names of Yinghe Hu, Michael L. McCaleb, Brian T. Bloomquist, Jaime R. Flores-Riveros, and Linda J Cornfield; U.S. Pat. No. 5,968,819, issued Oct. 19, 1999 in the names of Christophe P. G. Gerald, Richard L. Weinshank, Mary W. Walker, and Theresa Branchek; and in U.S. Pat. No. 5,985,616, issued Nov. 16, 1999 in the names of Eric McFee Parker, Catherine Devine Strader, and Mark Stephen Rudinski, each of which is incorporated herein by reference for its teachings in regard to NPY receptor structure and function.

### BRIEF DESCRIPTION OF THE DRAWINGS

FIG. 1. Chimeric NPY receptors exhibit altered functional G-protein coupling characteristics—G-protein alpha subunit rank order of ligand-induced responses. Data is expressed as % maximal response and was derived by determining the maximal agonist stimulated % above basal stimulation for each receptor type, and normalizing all other data within that receptor type to the maximal (100%) value. The indicated NPY expression vector constructs were those directing the expression of the Y1 receptor cDNA of SEQ. ID. NO:1 (filled bars), the Y5 receptor DNA of SEQ. ID. NO:4 (open bars), the chimeric NPY5ΔY1CT receptor cDNA of SEQ. ID. NO:7 (vertical stripes), or the chimeric NPY5ΔY1IC3 receptor cDNA of SEQ. ID. NO:5 (horizontal stripes).

### DESCRIPTION OF THE SEQUENCE LISTINGS

SEQ ID NO:1. Human Y1 receptor DNA sequence.

SEQ ID NO:2. Human Y1 receptor amino acid sequence.

SEQ ID NO:3. Rat Y1 receptor amino acid sequence.

SEQ ID NO:4. Human Y5 receptor DNA sequence.

SEQ ID NO:5. Human NPY5ΔY1IC3 chimera DNA sequence.

SEQ ID NO:6. Human NPY5ΔY1IC3 chimera amino acid sequence.

SEQ ID NO:7. Human NPY5ΔY1CT chimera DNA sequence.

SEQ ID NO:8. Human NPY5ΔY1IC3/ΔY1CT chimera DNA sequence.

SEQ ID NO:9. Human NPY5ΔY1CT chimera amino acid sequence.

SEQ ID NO:10. Human NPY5ΔY1IC3/ΔY1CT chimera amino acid sequence.

SEQ ID NO:11. Amino acid sequence of the His₆ₓ epitope.

SEQ ID NO:12. Amino acid sequence of the FLAG epitope.

SEQ ID NO:13. Human Y5 receptor amino acid sequence.

SEQ ID NO:14. 5′ Y5 primer.

SEQ ID NO:15. 3′ Y5 primer.

SEQ ID NO:16. HY1L3S sense oligo.

SEQ ID NO:17. HY1L3AS anti-sense oligo.

SEQ ID NO:18. HY1R1 forward primer (creates EcoR1 site).

SEQ ID NO:19. HY5R1 reverse primer (creates EcoR1 site).

SEQ ID NO:20. Dog NPY5ΔY1IC3 chimera.

SEQ ID NO:21. Dog NPY5ΔY1IC3/ΔY1CT chimera.

SEQ ID NO:22. Mouse NPY5ΔY1CT chimera.

SEQ ID NO:23. Rat NPY5ΔY1IC3 chimera.

SEQ ID NO:24. Rat NPY5ΔY1CT chimera.

SEQ ID NO:25. Rat NPY5ΔY1IC3/Y1CT chimera.

SEQ ID NO:26. Pig NPY5ΔY1IC3 chimera.

SEQ ID NO:27. Pig NPY5ΔY1IC3/ΔY1CT chimera.

SEQ ID NO:28. NPY5 forward primer hY5-45F.

SEQ ID NO:29. NPY5 reverse primer hY5-1450R.

SEQ ID NO:30. African Green Monkey NPY5 DNA sequence.

SEQ ID NO:31. African Green Monkey NPY5 amino acid sequence.

### SUMMARY OF THE INVENTION

It is an object of the present invention to provide novel chimeric NPY receptors. Preferably these receptors display the ligand binding pharmacological characteristics typical of Y5 receptors while mediating signal transduction effects typical of Y1 receptors (preferably involving G-protein coupling typical of Y1 receptors). It is an additional object to provide cells expressing such chimeric NPY receptors. Preferably these chimeric receptor-expressing cells provide a source of chimeric receptors (typically in the form of the cells themselves or in the form of isolated membrane preparations) that are adapted for use in robust assays of either or both of receptor binding and receptor function (e.g., receptor G-protein subunit binding or receptor signal transduction). Particularly preferred receptors can be expressed at higher levels than native (non-chimeric, non-mutant) Y5 receptors, and particularly preferred cells express such receptors at such higher levels.

It is a further object of the invention to provide assays for identifying compounds that specifically bind to NPY5 receptors. Such assays comprise contacting a compound to be tested with cells or isolated membranes of the invention and detecting the binding of the compounds to the cells.

The invention also deals with a method of treating a condition in a subject where the condition is, for example, an eating disorder, a siezure disorder, a blood pressure disorder, a locomoter disorder or an anxiety disorder. The method includes administering to the subject an effective amount of a composition comprising a compound identified by the aforementioned assays.

To these ends, this invention first provides chimeric NPY receptor proteins comprising a recipient NPY5 receptor comprising at least one domain substitution wherein the substitution comprises the replacement of one or both of the third intracellular loop domain and the C-terminal intracellular domain. The substituted donor domains are derived from a different type of NPY receptor (e.g., a Y1 receptor, a Y2 receptor, or a Y4 receptor, the “donor receptor”) than the recipient NPY5 receptor. Each donor NPY receptor preferably comes from the same class of animal, preferably from the same order of animal, more preferably from the same family of animal, yet more preferably from the same genus of animal, and most preferably from the same species of animal as the recipient NPY5 receptor is obtained from. Where at least two domains are substituted, each substituted donor domain may be obtained from the same or a different species of animal as the other, preferably all are from the same species of animal and from the same type of donor NPY receptor.

In this embodiment, each fragment of a substituted recipient domain of the recipient Y5 receptor is an intracellular domain consisting essentially of a contiguous length of at least about 50% the length of the entire recipient Y5 receptor domain in which the substitution is being made. In this embodiment, this NPY5 fragment is deleted and replaced by a corresponding fragment, i.e., one with termini located at about the same number of amino acid residues (e.g., within plus or minus 10%, preferably within plus or minus 5%, most preferably within plus or minus 2% of the number of amino acid residues in the entire corresponding donor domain) from the adjacent end of each adjacent donor NPY receptor TM domain (e.g., the fifth and sixth TM domains or the seventh TM domain) as each terminus of the deleted and replaced (recipient) fragment of the recipient Y5 receptor is located from its nearest (adjacent) recipient NPY5 receptor TM domain. Preferably the resulting domain of the chimeric receptor has 1) the same number of amino acids as the corresponding donor NPY receptor domain or 2) the same number of amino acids as the corresponding recipient NPY5 receptor domain, or, 3) a number of amino acids intermediate between 1) and 2). Such domain fragments may have each terminus (independently from any other terminus) located within an adjacent TM domain (except, of course, for the C-terminus of a C-terminal intracellular domain fragment) or located within the substituted domain.

Preferably all domains for the chimeric receptor other than the substituted domains are complete and contiguous with each other, so that the resulting chimeric receptor has the same sequence (starting at the N-terminus of the chimeric receptor) as the recipient receptor from the N-terminus of the recipient receptor to the C-terminus of the second extracellular domain and from the N-terminus of the third extracellular domain to the N-terminus of the seventh TM domain.

In a first aspect, the invention provides a chimeric NPY receptor protein having the amino acid sequence of an NPY5 receptor protein except that a third intracellular loop domain fragment of the Y5 receptor recipient is replaced by a third intracellular loop domain fragment of another (donor) NPY receptor.

In another aspect this invention provides a chimeric NPY receptor protein having the amino acid sequence of an NPY5 receptor protein except that a the C-terminal intracellular (hydrophilic) domain fragment of this protein is replaced by a corresponding fragment of the corresponding domain of another (donor) NPY receptor.

In a third aspect the invention provides a chimeric NPY receptor having the amino acid sequence of an NPY5 receptor protein except that the chimeric receptor includes both of the NPY receptor protein fragment substitutions described in the two preceding paragraphs.

In additional aspects, the invention provides nucleic acid molecules (preferably isolated nucleic acid molecules) encoding the chimeric NPY receptors of the invention as well as cells (preferably animal cells and preferably cultured cells) comprising expression vectors comprising such nucleic acid molecules and thereby expressing the chimeric NPY receptors of the invention. Preferably these cells bind higher levels per cell of an NPY ligand (e.g., NPY or PYY) than do matched control cells comprising matched control expression vectors and thereby expressing matched native (non-chimeric, non-mutant) NPY5 receptors. The invention further provides a novel monkey NPY5 receptor and chimeras comprising NPY5 domains of this monkey receptor.

### DETAILED DESCRIPTION OF PREFERRED EMBODIMENTS OF THE INVENTION

### Nucleic Acid Molecules

This invention provides nucleic acid (NA) molecules (including fragments, e.g., PCR products or restriction fragments) that encode chimeric NPY receptor proteins, preferably chimeric Y5/Y1 receptor proteins. Preferably the NA molecules are clones and are isolated NA molecules. In accordance with the invention, these NA molecules include genomic DNA molecules, cDNA molecules, RNA molecules, and modified analogs of such NA molecules, such as phosphorthioate derivatives and the like.

In a first aspect, the invention provides NA molecules (e.g., a clone) encoding a chimeric NPY receptor protein having the amino acid sequence of an NPY5 receptor protein (preferably a human Y5 receptor protein) except that intracellular loop 3 of this protein has been replaced by intracellular loop 3 of an NPY1 receptor protein (preferably a human Y1 receptor protein). In other words, the encoded chimeric protein is structurally characterized as comprising a single polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain, an NPY1 third intracellular loop domain, an NPY5 sixth TM domain, an NPY5 third extracellular loop domain, an NPY5 seventh TM domain, and an NPY5 C-terminal intracellular domain. Preferably the donor Y1 moiety in the location of the third intracellular loop domain of the chimeric receptor is a contiguous Y1 sequence that comprises at least one extension partially or completely into one or both of the immediately adjacent TM domains of the donor Y1 receptor, replacing the corresponding sequence(s) of the recipient Y5 receptor. In certain preferred embodiments, the Y1 moiety in the location of the third intracellular loop domain does not comprise the entire third intracellular loop domain, but only a substantial (at least about 15, preferably at least about 20, and most preferably at least 21 amino acids in length) contiguous portion of the entire donor Y1 third intracellular loop domain. In such an embodiment, the replaced portion of intracellular loop 3 of the recipient Y5 receptor includes the amino acids encoded by nucleotides no. 752-1129 of SEQ ID NO:4. Thus in a preferred embodiment the invention provides isolated NA molecules (e.g., an isolated clone) comprising a cDNA sequence (SEQ ID NO:5) encoding the amino acid sequence of SEQ ID NO:6, referred to as NPY5ΔY1IC3. In a related embodiment, the NA molecule of SEQ ID NO:4 has been altered by the deletion of a fragment consisting essentially of nucleotides 752-1129 of SEQ ID NO:4 and its replacement (in the same in-frame coding orientation) by a fragment consisting essentially of nucleotides 902-964 of SEQ ID NO:1.

In a separate embodiment the invention provides a chimeric NPY receptor protein comprising the amino acid sequence of the N-terminal domain, intracellular loops, extracellular loops and TM domains of a recipient NPY5 receptor protein (preferably a human Y5 receptor protein) and the C-terminal intracellular domain of a donor NPY1 receptor protein (preferably a human Y1 receptor protein). In other words, the encoded chimeric protein is structurally characterized as comprising a single polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain, an NPY5 third intracellular loop domain, an NPY5 sixth TM domain, an NPY5 third extracellular loop domain, at least part of an NPY5 seventh TM domain, and an NPY1 C-terminal intracellular domain.

In certain aspects of the invention, the Y1 C-terminal intracellular domain is a contiguous Y1 sequence that extends partially or completely into the immediately adjacent TM domain of Y1, replacing the corresponding sequence of the Y5 receptor. In certain preferred embodiments, the Y1 moiety in the location of the C-terminal intracellular domain does not comprise the entire C-terminal intracellular domain, but only a substantial (at least about 40, preferably at least about 50, and most preferably at least 57 amino acids in length) contiguous portion of the entire Y1 C-terminal intracellular domain, preferably the Y1 moiety extends to and includes the C-terminal amino acid of Y1 (i.e., the C-terminus of the Y1 C-terminal intracellular domain). In another preferred embodiment the donor C-terminal domain in the chimeric receptor includes all of the amino acids from the C-terminal end of the donor seventh TM domain to the C-terminus of the donor receptor.

More preferably the replaced Y5 recipient C-terminal domain includes the amino acids encoded by nucleotides no. 1343-1384 of SEQ ID NO:4. In a preferred embodiment the invention provides isolated NA molecules comprising the cDNA sequence of SEQ ID NO:7 (NPY5ΔY1CT). In a related embodiment, the NA molecule of SEQ ID NO:4 has been altered by the deletion of a fragment consisting essentially of nucleotides 1343-1384 of SEQ ID NO:4 and its replacement (in the same in-frame coding orientation) by a fragment consisting essentially of nucleotides 1178-1351 of SEQ ID NO: 1.

In another embodiment the invention provides isolated NA molecules encoding the amino acid sequence of a recipient NPY5 receptor protein (preferably a human Y5 receptor protein) except that intracellular loop 3 of this protein has been replaced intracellular loop 3 of an NPY1 receptor protein (preferably a human Y1 receptor protein) and the C-terminal intracellular domain of this protein has been replaced by the C-terminal intracellular domain of an NPY1 receptor protein (preferably the same Y1 receptor protein as that providing the third intracellular loop domain, preferably a human Y1 receptor protein). In other words, the encoded chimeric protein is structurally characterized as comprising a single polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, at least part of an NPY5 fifth TM domain, an NPY1 third intracellular loop domain, at least part of an NPY5 sixth TM domain, an NPY5 third extracellular loop domain, at least part of an NPY5 seventh TM domain, and an NPY1 C-terminal intracellular domain.

Intracellular loop 3 and the C-terminal domain in this chimeric receptor protein are as described above. In a preferred embodiment the invention provides NA molecules comprising the cDNA sequence of SEQ ID NO:8 (encoding NPY5ΔY1IC3/ΔY1CT). In a related embodiment, the NA molecule of SEQ ID NO:4 has been altered by the deletion of a fragment consisting essentially of nucleotides 752-1129 of SEQ ID NO:4 and its replacement (in the same in-frame coding orientation) by a fragment consisting essentially of nucleotides 902-964 of SEQ ID NO:1 and the NA molecule of SEQ ID NO:4 has been further altered by the deletion of a fragment consisting essentially of nucleotides 1343-1384 of SEQ ID NO:4 and its replacement (in the same in-frame coding orientation) by a fragment consisting essentially of nucleotides 1178-1351 of SEQ ID NO:1.

This invention also includes NA molecules (preferably isolated, preferably a clone thereof) encoding an amino acid sequence selected from the group consisting of SEQ ID NO:6, SEQ ID NO:9, SEQ ID NO:10, SEQ ID NO:20, SEQ ID NO:21, SEQ ID NO:22, SEQ ID NO:23, SEQ ID NO:24, SEQ ID NO:25, SEQ ID NO:26, SEQ ID NO:27 and SEQ ID NO:31, as well as NA molecules (preferably isolated, preferably a clone thereof) comprising a nucleic acid sequence selected from the group consisting of SEQ ID NO:5, SEQ ID NO:7, SEQ ID NO:8, and SEQ ID NO:30.

It will be apparent to those skilled in the art that, due to the degeneracy of the genetic code, substituting 1 or more redundant codons can create numerous variants of the described NA molecules without changing the amino acid sequence of the encoded protein product. Additionally, sequence changes may be made in the non-coding regions of NA sequences without altering the amino acid sequence of the encoded protein product.

Also within the scope of the present invention are certain changes to DNA and cDNA sequences encoding the chimeric NPY receptor proteins of SEQ ID NO:6, SEQ ID NO:9, SEQ ID NO:10, SEQ ID NO:20, SEQ ID NO:21, SEQ ID NO:22, SEQ ID NO:23, SEQ ID NO:24, SEQ ID NO:25, SEQ ID NO:26, and SEQ ID NO:27. These include in-frame additions of NA sequences encoding short amino acid sequences useful as antibody recognition (tag) sequences. Such amino acid sequences are well known in the art, and include, but are not limited to the His-6x (hexa-histidine or His tag) epitope (SEQ ID NO:11) which chelates metals such as nickel (facilitating protein purification via metal chelation chromatography) and is specifically bound by Monoclonal Anti-polyhistidine Clone HIS-1 antibody (Sigma, St. Louis No. H1029), and the FLAG epitope (SEQ ID NO:12) which is specifically bound by the FLAG-M2 monoclonal antibody (Sigma, St. Louis No. F3165). Techniques for making such modifications are also well known in the art, and may be readily carried out using routine methods or by using prepared kits, for example, the Sigma Mammalian FLAG Expression Kits (Sigma, St. Louis, e.g., Nos. FL-MA and FL-MC). Preferably the fusions are made as in-frame amino- (N-) or carboxy- (C-) terminal fusions. C-terminal fusions are preferred as generally being less prone to interfering with efficient membrane insertion of the fusion protein.

A tagged fusion protein may be purified using an antibody specific for the tag, e.g., by affinity chromatography. Such purification procedures will typically require detergent extraction unless the protein to be purified is not inserted in a membrane. Such purified proteins are useful as antigens for the preparation of receptor-specific antibodies, in which case the retention of receptor signal transduction function is typically of no consequence. Additional embodiments of NA molecules of the invention are those encoding the polypeptides of the invention discussed below (particularly those that have not been previously described herein; see, e.g., A) B) and C)).

### Polypeptides

The present invention provides chimeric NPY receptor polypeptides (preferably isolated polypeptides) encoded by the NA molecules described above. In certain preferred embodiments, the chimeric polypeptides of the invention have the amino acid sequence of SEQ ID NO:6, SEQ ID NO:9, or SEQ ID NO:10. The amino acid sequence of SEQ ID NO:6 is the protein product encoded by SEQ ID NO:5, the amino acid sequence of SEQ ID NO:9 is the protein product encoded by SEQ ID NO:7, and the amino acid sequence of SEQ ID NO:10 is the protein product encoded by SEQ ID NO:8. In certain additional preferred embodiments, the chimeric polypeptides of the invention have the amino acid sequence of SEQ ID NO:20, SEQ ID NO:21, SEQ ID NO:22, SEQ ID NO:23, SEQ ID NO:24, SEQ ID NO:25, SEQ ID NO:26, or SEQ ID NO:27. The invention also encompasses chimeric NPY receptor proteins having amino acid sequences that differ from these, as described above in the discussion of NA molecules.

In additional embodiments, the invention provides:

A) A chimeric receptor protein comprising a single continuous polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain optionally substituted at the C-terminal end of the domain with up to 20 amino acids of a contiguous corresponding C-terminal portion of an NPY1 fifth TM domain (when so substituted, such an optionally substituted TM domain being referred to as a “hybrid Y5/Y1 TM domain”), a third intracellular loop domain comprising at least a substantial contiguous portion of an NPY1 third intracellular loop domain, an NPY5 sixth TM domain optionally substituted at the N-terminal end of the domain with up to 20 amino acids of a contiguous corresponding N-terminal portion of an NPY1 sixth TM domain to yield a hybrid Y5/Y1 TM domain, an NPY5 third extracellular loop domain, an NPY5 seventh TM domain, and an NPY5 C-terminal intracellular domain: provided that when either the fifth or sixth TM domain is a hybrid Y5/Y1 TM domain, the portion of an NPY1 third intracellular loop domain is a portion that is contiguous with the corresponding TM domain in native NPY1, and that when both the fifth and sixth TM domains are hybrid Y5/Y1 TM domains, the portion of an NPY1 third intracellular loop domain is an entire NPY1 third intracellular loop domain. Preferably this chimeric receptor protein polypeptide chain consists of about from 335 to 365 amino acids. More preferably this chimeric receptor protein polypeptide chain consists of from 341 to 352 amino acids optionally extended by the addition of a tag sequence of about 6 to 8 amino acids. Most preferably this chimeric receptor protein polypeptide chain consists of 341, 350, or 352 amino acids, each optionally extended by the addition of a tag sequence of about 6 to 8 amino acids.

B) A chimeric receptor protein comprising a single continuous polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain, an NPY5 third intracellular loop domain, an NPY5 sixth TM domain, an NPY5 third extracellular loop domain, an NPY5 seventh TM domain optionally substituted at the C-terminal end of the domain with up to 20 amino acids of a contiguous corresponding C-terminal portion of an NPY1 seventh TM domain to yield a hybrid Y5/Y1 TM domain, and at least a substantial portion of an NPY1 C-terminal intracellular domain: provided that when the seventh TM domain is a hybrid Y5/Y1 TM domain, the portion of an NPY1 C-terminal intracellular domain is a portion that (both in the native NPY1 donor receptor and in the resulting chimeric receptor) is contiguous with the seventh TM domain. Preferably this chimeric receptor protein polypeptide chain consists of about from 485 to 516 amino acids. More preferably this chimeric receptor protein polypeptide chain consists of from 488 to 508 amino acids optionally extended by the addition of a tag sequence of about 6 to 8 amino acids. Most preferably this chimeric receptor protein polypeptide chain consists of 488, 499, or 508 amino acids, each optionally extended by the addition of a tag sequence of about 6 to 8 amino acids.

C) A chimeric receptor protein comprising a single continuous polypeptide chain comprising, in N-terminal to C-terminal order, an NPY5 N-terminal extracellular domain, an NPY5 first TM domain, an NPY5 first intracellular loop domain, an NPY5 second TM domain, an NPY5 first extracellular loop domain, an NPY5 third TM domain, an NPY5 second intracellular loop domain, an NPY5 fourth TM domain, an NPY5 second extracellular loop domain, an NPY5 fifth TM domain optionally substituted at the C-terminal end of the domain with up to 20 amino acids of a contiguous corresponding C-terminal portion of an NPY1 fifth TM domain to yield a hybrid Y5/Y1 TM domain, a third intracellular loop domain comprising at least a substantial contiguous portion of an NPY1 third intracellular loop domain, an NPY5 sixth TM domain optionally substituted at the N-terminal end of the domain with up to 20 amino acids of a contiguous corresponding N-terminal portion of an NPY1 sixth TM domain to yield a hybrid Y5/Y1 TM domain, an NPY5 third extracellular loop domain, an NPY5 seventh TM domain optionally substituted at the C-terminal end of the domain with up to 20 amino acids of a contiguous corresponding C-terminal portion of an NPY1 seventh TM domain to yield a hybrid Y5/Y1 TM domain, and at least a substantial portion of an NPY1 C-terminal intracellular domain: provided that when either the fifth or sixth TM domain is so optionally substituted, the portion of an NPY1 third intracellular loop domain is a portion that is contiguous with the optionally substituted TM domain in native NPY1, that when both the fifth and sixth TM domains are so optionally coupled, the portion of an NPY1 third intracellular loop domain is an entire NPY1 third intracellular loop domain, and that when the seventh TM domain is so optionally substituted, the portion of an NPY1 C-terminal intracellular domain is a portion that (both in the native NPY1 donor receptor and in the resulting chimeric receptor) is contiguous with the seventh TM domain. Preferably this chimeric receptor protein polypeptide chain consists of about from 380 to 405 amino acids. More preferably this chimeric receptor protein polypeptide chain consists of from 383 to 395 amino acids optionally extended by the addition of a tag sequence of about 6 to 8 amino acids. Most preferably this chimeric receptor protein polypeptide chain consists of 383, 394, or 395 amino acids, each optionally extended by the addition of a tag sequence of about 6 to 8 amino acids.

### Expression Systems

Expression systems that may be used in the practice of certain aspects of the invention include but are not limited to insect cell systems infected with recombinant virus expression vectors (for example, baculovirus) comprising the NA molecules of the invention and mammalian cell systems (for example, COS, CHO, BHK, 293, VERO, HeLa, MDCK, WI38, and NIH 3T3 cells) harboring recombinant expression constructs comprising the NA molecules of the invention. Such mammalian vectors should contain promoters, preferably derived from the genome of mammalian cells (for example, the metallothionein promoter) or from mammalian viruses (for example, the adenovirus late promoter, the CMV promoter and the vaccinia virus 7.5K promoter). Such promoters should be operatively linked to a NA fragment of the invention.

Another preferred expression system is an amphibian oocyte comprising RNA molecules of the invention generated, preferably via an in vitro transcription system, using an expression vector of the invention. Preferably the amphibian is a frog, most preferably the African clawed frog, Xenopus laveis.

An expression vector of the invention is a vector for recombinant expression of a chimeric receptor protein of the invention, wherein a nucleic acid of the invention is operatively linked to at least one regulatory element (wherein a regulatory element is a nucleic acid sequence that directs the expression of adjacently linked coding sequences) in the appropriate orientation for expression. Such a vector is preferably a plasmid or viral vector.

A cell of the invention is one comprising an expression vector of the invention, and thereby expressing at least one chimeric NPY receptor of the invention.

An insect system utilizing a baculovirus such as Autographa califormica nuclear polyhedrosis virus (AcNPV) can be used to express the recombinant receptors of the invention. The virus grows in insect cells such as Spodoptera frugiperda cells (e.g. Sf9). The coding sequence encoding the chimeric NPY receptor of the invention is typically inserted (e.g., ligated) into non-essential regions of the virus (for example into the polyhedrin gene) and placed under control of an AcNPV promoter (for example the polyhedrin promoter). Preferably the successful introduction of the insert will result in inactivation of a viral gene. For example, when targeted into the polyhedrin gene, the successful incorporation of the insert will inactivate that gene and result in production of non-occluded recombinant virus (i.e., virus lacking the proteinaceous coat coded for by the polyhedrin gene). The resulting recombinant viruses are then used to infect insect cells, preferably Spodoptera frugiperda cells, in which the inserted coding sequence is expressed (see, e.g., Smith et al., J. Virol., 46:584, 1983).

In mammalian host cells, a number of expression systems, including viral-based expression systems, may be utilized. In those aspects of the invention involving an animal comprising cells comprising an insert encoding a chimeric receptor of the invention whereby cells of the animal express a chimeric receptor of the invention, i.e., a transgenic animal of the invention, non-viral expression systems are generally preferred.

In cases where an adenoviral vector is used as an expression vector, the nucleic acid molecule of the invention may be ligated to an adenovirus transcription/translation control complex such as the late promoter and tripartite leader sequence. This recombinant gene may then be inserted in the adenovirus genome by in vitro or in vivo recombination. Insertion in a non-essential region of the viral genome (for example, region E1 or E3) will result in a recombinant virus that is viable and capable of expressing a chimeric NPY receptor gene product of the invention in infected hosts (for example, see Logan and Shenk, Proc. Natl. Acad. Sci. USA, 81:3655-3659, 1984). Specific initiation signals may also be required for efficient translation of inserted nucleic acid molecules. These signals include the ATG initiation codon and adjacent sequences such as ribosome binding sites, which signals and their uses are well known to those of skill in the art. The efficiency of expression may be enhanced by the inclusion of appropriate transcription enhancer elements, transcription terminators, etc. (see, e.g., Bittner et al., Methods in Enzymol., 153:516-544, 1987). A preferred mammalian expression vector is the PCDNA3.1 vector available from INVITROGEN Corporation, Carlsbad, Calif.

A preferred expression vector for insertion of a nucleic acid fragment of the invention for expression thereof in amphibian oocytes is the PBLUESCRIPT SK⁻ vector available from STRATAGENE Cloning Systems, La Jolla, Calif. Typically such vectors are used to generate chimeric-receptor-encoding RNAs in in-vitro transcription systems, which RNAs are then injected into the oocytes to induce expression of the chimeric receptor of the invention.

While transient expression systems are within the scope of the invention, long-term expression of recombinant proteins, particularly in cultured mammalian cells, is also within its scope. For such long-term expression (which is preferably adapted for high-level expression) stable expression is preferred. Host cells can be transformed with a vector comprising, in appropriate orientations for expression, appropriate expression control elements (for example, promoter, enhancer sequences, transcription terminators, and polyadenylation signals), and (preferably also in functional linkage to expression elements) a selectable marker. Following the introduction of the vector (often following incubation in a non-selective medium to allow for recovery from the stress of vector introduction), engineered cells may be grown in a selective medium. The selectable marker in the recombinant plasmid confers resistance to the selection and allows cells to stably integrate the plasmid into their chromosomes and grow to form foci that in turn can be cloned and expanded into cell lines. A number of selection systems can be used. For example, the hypoxanthine-guanine phosphoribosyl-transferase (Szybalska and Szybalski, Proc. Natl. Acad. Sci. USA, 48:2026, 1962), adenine phosphoribosyltransferase (Lowy, et al., Cell, 22:817, 1980) and herpes simplex virus thymidine kinase (Wigler, et al., Cell, 11:223, 1977) genes can be employed in hgprt⁻, aprt⁻ or tk⁻ cells, respectively. Also, anti-metabolite resistance can be used as the basis of selection for genes such as: dhfr, which confers resistance to methotrexate (Wigler et al., Proc. Natl. Acad. Sci. USA, 77:3567, 1980; O'Hare et al., Proc. Natl. Acad. Sci. USA, 78:1527, 1981); gpt, which confers resistance to mycophenolic acid (Mulligan and Berg, Proc. Natl. Acad. Sci. USA, 78:2072, 1981); neo, which confers resistance to the aminoglycoside G-418 (Colberre-Garapin et al., J. Mol. Biol., 150:1, 1981); hygro, which confers resistance to hygromycin (Santerre et al., Gene, 30:147, 1984); and puro, which confers resistance to puromycin (Ausubel, et al., eds., Current Protocols in Molecular Biology, John Wiley & Sons, 1999).

### Isolated Membranes of Recombinant Cells

In certain of its aspects the present invention provides a preparation comprising isolated membranes of the recombinant cells of the invention (also referred to herein and in the claims as a preparation of recombinant membranes). Preferably, the isolated membranes should exhibit neuropeptide Y binding activity that is at least 2-fold greater, preferably 10-fold greater and more preferably at least 20-fold greater than that exhibited by control membranes isolated from a control host cell (e.g., a cell of the same cell line used to prepare the recombinant cell of the invention that does not contain any vector, or contains a control vector that does not encode an NPY receptor). Preferred membranes contain at least 0.1 pmol, more preferably at least 1 pmol, and most preferably at least 5 pmol of chimeric NPY receptor protein per mg of total membrane protein. Membranes can be isolated by any suitable method, such as any of the membrane preparation methods that are routinely used in the art.

### Assays

The assays of the present invention involve contacting a compound to be tested with cells or isolated membranes of the invention and detecting the binding of the compounds to the cells or membranes. These assays are useful, e.g., for identifying or characterizing compounds that specifically bind to NPY5 receptors, which compounds are useful, e.g., as tools for receptor mapping and as pharmaceutical agents.

Assays for detecting compounds that interact with NPY receptors are well known in the art, and can be readily adapted to be assays of the invention by using (as substrates for receptor binding) cells or membranes of the invention, rather than those previously known in the art. Such assays typically involve measuring responses of receptors to being contacted with a compound to be tested (functional assays) or measuring the capacity of a compound to be tested to displace the receptor binding of a labeled (e.g., radiolabeled) compound known to bind to such a receptor (binding assays). An exemplary binding assay of the invention is set forth below as Example 5. In such an assay of the invention, a compound to be tested is used as a cold displacer. An exemplary functional assay of the invention is set forth below as Example 6. In such an assay of the invention, a compound to be tested is used as was the agonist in Example 6. Other functional assays of the invention use cells of the invention as substrates and measure cellular responses to being contacted with compounds to be tested.

The aforementioned assays, which identify test compounds which interact with the chimeric receptors and modulate intracellular signalling, can be used to diagnose or treat conditions including, but not limited to, obesity, high/low blood pressure, anxiety, epilepsy, Huntington's, and Parkinson's.

Pharmaceutically useful compositions comprising modulators of chimeric receptor activity, identified from the screening assays, may be formulated. Such therapeutic or diagnostic compositions may be administered to a subject in amounts effective to treat or diagnose disorders.

### EXAMPLES

### Example 1

### DNA Clones Encoding NPY Receptors

Human Y5 receptor was cloned from genomic DNA using a 5′ Primer (SEQ ID NO:14) TTTTGGTTGCTGACAAATGTC and a 3′ Primer (SEQ ID NO:15)

CCTTGGTAAACAGTGAGAATTATTAC. The full length PCR product was initially cloned into the vector pCR 2.1 (Invitrogen, Carlsbad, Calif.) and then subcloned into pBluescript SK Minus (pBSSKM, Stratagene, La Jolla Calif.) to yield clone pNN32. A pBSSKM clone encoding a 5′ truncated form of the Y5 receptor was made which deleted the 5′ end of the coding region to the Nco I site (located at about residues 508-513 of SEQ ID NO:4). This was designated pNN39.

A cDNA encoding the human Y1 Receptor (Genbank Accession number M88461, SEQ ID NO:1) was obtained from Claes Wahlestedt (New York Hospital, Cornell Medical Center, Dept. of Neurology and Neuroscience) and bases 197 to 1433 of SEQ ID NO:1 were subcloned in a series of routine steps into pBSSKM, the resulting clone designated pNN22.

For an NPY5/Y1 IC loop 3 chimera, pNN39 was digested with Pst I (located at about residues 748-753 of SEQ ID NO:4) and Bgl II (located at about residues 1130-1135 of SEQ ID NO:4) removing bases 753 to 1130 of SEQ ID NO:4.

The portion of IC loop 3 from bases 903-964 (TACGCCTAAAAAGGAGAAACAACATGATGGACAAGATGAGAGACAATAAGT ACAGGTCCAGT) of SEQ ID NO:1, corresponding to amino acids 236-256 (IRLKRRNNMMDKMRDNKYRSS) of SEQ ID NO:2, was inserted into Y5 using the HY1L3S sense oligo (SEQ ID NO:16) and the HY1L3AS antisense oligo (SEQ ID NO:17). A reaction mixture containing the 2 oligos was heated to 100 degrees C. and allowed to cool slowly to anneal the oligos. The double stranded annealing product was then ligated into the Pst I-Bgl II digested pNN39 to yield plasmid pPB1. The pPB1 insert was then reintroduced into the full-length human Y5 gene (pNN32) at the Cel 2 site (located at about residues 619-625 of SEQ ID NO:4) and the resulting plasmid was designated pNN42. The coding region of the insert of this vector is found in SEQ ID NO:5, hNPY5ΔY1IC3, and encodes the amino acid sequence of SEQ ID NO:6.

To add the Y1 C-terminus to Y5, an Eco RI site was added to each gene. For Y1, bases 1173 to 1178 (ACTTCC) of SEQ ID NO:1 were mutated to create an Eco R1 site via PCR from forward primer HY1R1 (SEQ ID NO:18) to a T3 primer (priming from the multiple cloning site —“MCS”—of pBSSKM). The Y1 3′ tail was then isolated by digesting with Eco RI and Xba I (which latter enzyme cuts out the Y1 3′ tail in the MCS of pBSSKM). For Y5, bases 1338 to 1343 (GGATTA) of SEQ ID NO:4 were mutated using the PCR reverse primer HY5R1 (SEQ ID NO:19). This primer was paired with a forward primer corresponding to bases 527-551 (GCTACTGTCTGGACACTAGGTTTTG) of SEQ ID NO:4, and PCR carried out with pNN32 as template. The resulting PCR band was cut from the unique Pst I site in the PCR product to the introduced Eco RI site.

pNN39 was then opened Pst I to Xba from the MCS of pBSSKM and the mutated Y5 segment Pst1 to Eco RI was mixed with the mutated Y1 3′ fragment Eco RI to Xba from the MCS to set up a three-way ligation. The resulting mutated gene fragment was then introduced into the full-length Y5 gene at the Bgl II site as a Bgl II-Xba I fragment to yield construct pNN43. The coding region of the insert of this construct is found in SEQ ID NO:7, NPY5ΔY1CT, and encodes the amino acid sequence of SEQ II) NO:9.

The IC loop 3+CT tail exchange was obtained by combining the above 2 mutant genes in the following manner. Full length hY5 (pNN32) was digested with Cel 2 (located at about residues 619-625 of SEQ ID NO:4) and Xba in the vector MCS. The loop 3 mutation pNN42 fragment Cel II to Bgl II was combined with the CT mutation pNN43 fragment Bgl II to Xba (the Xba is in the MCS) resulting in pNN44. The coding region of the insert of this vector is found in to SEQ ID NO:8, hNPY5ΔY1IC3/ΔYCT, and encodes the amino acid sequence of SEQ ID NO: 10.

Each of the three chimeric NPY5/NPY1 receptors was then digested with Kpn I and Xba I and separately subcloned into the commercial expression vector pcDNA 3.1+(Invitrogen, Carlsbad, Calif.) for expression in mammalian cells and into the commercial expression vector pBacPAK9 (CLONTECH, Palo Alto, Calif.) for expression in SF9 cells.

### Example 2

### Additional NPY Receptors

Additional examples of chimeric NPY receptors of the invention are set forth in the sequence listings as follows. The canine NPY receptor chimeras cNPY5ΔcY1IC3 (SEQ ID NO:20) and (cNPY5ΔcY1IC3/ΔcY1CT SEQ ID NO:21). The murine NPY receptor chimera mNPY5ΔmY1CT (SEQ ID NO:22). The rat NPY receptor chimeras rNPY5ΔrY1IC3 (SEQ ID NO:23), rNPY5ΔrY1CT (SEQ ID NO:24), and (rNPY5ΔrY1IC3ΔrY1CT SEQ ID NO:25). The porcine NPY receptor chimeras pNPY5ΔpY1IC3 (SEQ ID NO:26) and pNPY5ΔpY1CTΔpY1CT (SEQ ID NO:27).

A novel African Green Monkey (AGM) NPY5 receptor was cloned via PCR from COS cell DNA using the forward primer hY5-45F (SEQ ID NO:28) and the reverse primer hY5-1450R (SEQ ID NO:29), both of which primers were designed using the human NPY5 DNA sequence of SEQ ID NO:4. The forward primer, hY5-45F, comprises 5 bases encoding (with the addition of a sixth base at the 3′ end) the first two amino acids of human NPY5. The complete sequence of the AGM NPY5 PCR product is set forth as SEQ ID NO:30 and the amino acid sequence encoded thereby is set forth as SEQ ID NO:31. This amino acid sequence (SEQ ID NO:31) differs from the amino acid sequence of human Y5 (SEQ ID NO: 13) in having an arginine instead of a lysine at position 273, an isoleucine instead of a serine at position 275 and a methionine instead of a valine at position 447.

### Example 3

### Baculoviral Preparations

Each Baculoviral expression vector was co-transfected along with BACULOGOLD DNA (BD PHARMINGEN, San Diego, Calif.) into Sf9 insect cells. The Sf9 cell culture supernatant was harvested three days post-transfection. The recombinant virus-containing supernatant was serially diluted in Hink's TNM-FH insect medium (JRH Biosciences, Kansas City) supplemented Grace's salts and with 4. 1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques were selected and harvested into 1 ml of insect medium for amplification. Each 1 ml volume of recombinant baculovirus (at passage 0) was used to infect a separate T25 flask containing 2×10⁶ Sf9 cells in 5 mls of insect medium. After five days of incubation at 27° C., supernatant medium was harvested from each of the T25 infections for use as passage 1 inoculum. Two of the seven recombinant baculoviral clones were then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml prep was harvested and plaque assayed for titer. The cell pellets from the second round of amplification were assayed by affinity binding as described below in Example 5 to verify recombinant receptor expression. A third round of amplification was then initiated using a multiplicity of infection (M.O.I.) of 0.1 to infect a liter of Sf9 cells. Forty hours post-infection the supernatant medium was harvested to yield passage 3 baculoviral stock and the cell pellet assayed for affinity binding. Titer of the passage 3 baculoviral stock was determined by plaque assay and an M.O.I. and Incubation Time Course experiment was carried out to determine conditions for optimal receptor expression. Results from the receptor optimization experiment show that an M.O.I. of 0.1 and a 72 hour incubation were the ideal infection parameters in order to achieve optimum Y5 receptor expression in up to 1 liter Sf9 cell infection cultures.

Log-phase Sf9 cells were infected with a stock of recombinant baculovirus (prepared as described for Y5, above) encoding either NPY5 (SEQ ID NO:13), NPY5 Y1IC3 (SEQ ID NO:6), or NPY5ΔY1CT (SEQ ID NO:9) followed by culturing in insect medium at 27° C. 72 hours post-infection, a sample of cell suspension was analyzed for viability by trypan blue dye exclusion, and the remaining Sf9 cells were harvested via centrifugation (3000 rpm/ 10 minutes/ 4° C.).

### Example 4

### Purified Membranes

Sf9 cell pellets prepared in Example 3 were resuspended in homogenization buffer (10 mM HEPES, 250 mM sucrose, 0.5 μg/ml leupeptin, 2 ,μ/ml Aprotinin, 200 μM PMSF, and 2.5 mM EDTA, pH 7.4) and homogenized using a POLYTRON homogenizer (setting 5 for 30 seconds). The homogenate was centrifuged (536×g/ 10 minutes/ 4° C.) to pellet the nuclei. The supernatant containing isolated membranes was decanted to a clean centrifuge tube, centrifuged (48,000×g/30 minutes, 4° C.) and resuspended in 30 ml homogenization buffer. This centrifugation and resuspension step was repeated twice. The final pellet was resuspended in ice cold Dulbecco's PBS containing 5 mM EDTA and stored at −80° C. in aliquots until needed. The protein concentration of the resulting membrane preparation was measured using the Bradford protein assay (Bio-Rad Laboratories, Hercules, Calif.). By this measure, a 1-liter culture of cells typically yielded 100-125 mg of total membrane protein.

### Example 5

### Radioligand Binding Assays for Modulators of Chimeric Receptors

Purified P2 membranes, prepared by the method given above in Example 4, were washed with PBS and resuspended by Dounce homogenization (tight pestle) in binding buffer (50 mM Tris-HCl, 5 mM KCl, 120 mM NaCl, 2 mM CaCl₂, 1 mM MgCl₂, 0.1% BSA, pH 7.4).

For saturation binding analysis, membranes (5-50 μg) were added to polypropylene tubes containing 0.010-0.500M [¹²⁵1]PYY (porcine, New England Nuclear Corp., Boston, Mass.; Sigma Biochemicals and Reagents 2000-2001; No. P5801). For evaluation of guanine nucleotide effects on receptor affinity, GTPγS was added to duplicate tubes at a final concentration of 50μM. Table I shows an [¹²⁵I]-PYY saturation summary with PYY binding kinetics and receptor expression levels for each receptor construct as indicated.

The data in Table I indicate that both chimeric constructs demonstrate equivalent or lower Kd, suggesting equivalent or higher receptor affinities for PYY, as compared with the native NPY5 receptor. The data also show that there is increased expression of both chimeric receptors on cell membranes.

For competition analysis (Table II), membranes (5-50 ,μg) were added to polypropylene tubes containing 0.050nM L[¹²⁵I]PYY (porcine). Cold displacers (“Peptide”) specifically human NPY 1-36, human NPY 3-36, human NPY 13-36, human D-Trp 32 NPY and human pancreatic polypeptide —“hPP”, all from American Peptide Co., Sunnyvale, Calif., were added to separate assays at concentrations ranging from 10⁻¹² M to 10⁻⁶M to yield a final volume of 0.250 mL. These peptides allow for the discrimination of specific NPY receptor pharmacological profiles. Nonspecific binding was determined in the presence of 1 μM NPY (human, American Peptide Co., Sunnyvale, Calif.) and accounted for less that 10% of total binding. Following a 2-hour incubation at room temperature, the reaction was terminated by rapid vacuum filtration. Samples were filtered over presoaked (in 1.0% polyethyeneimine for 2 hours prior to use) GF/C WHATMAN filters and rinsed 2 times with 5 mLs cold binding buffer without BSA. Remaining bound radioactivity was quantified by gamma counting. Kᵢ and Hill coefficient (“nH”) were determined by fitting the Hill equation to the measured values with the aid of SIGMAPLOT software (SPSS Inc., Chicago).

It is theorized, from the data in Table II, that changes in the amino acid sequences of receptor domains from those of native NPY5 may change the structural conformation of the receptor upon ligand binding thus affecting the receptor affinity for [¹²⁵I] PYY.

TABLE I

| NPY5 Receptor | | Kd (nM)* | Bmax (fmol/gm)* |
|---|---|---|---|
| NPY5 | | 0.183 ± .04 | 484 ± 295 |
| | +50 μM GTPγS | 0.398 ± .11 | 503 ± 295 |
| NPY5ΔY1IC3 | | 0.082 ± .02 | 1573 ± 816 |
| | +50 μM GTPγS | 0.110 ± .01 | 1555 ± 842 |
| NPY5Δγ1CT | | 0.207 ± .05 | 949 ± 175 |
| | +50 μM GTPγS | 0.332 ± .05 | 950 ± 71 |

*Average ± standard deviation

TABLE II

| | NPY5 | | NPY5ΔY1IC3 | | NPY5ΔY1CT | |
|---|---|---|---|---|---|---|
| DISPLACER | Ki (nM) | nH | Ki (nM) | nH | Ki (nM) | nH |
| HNPY 1-36 | 0.44 | 0.7 | 0.57 | 1.0 | 0.40 | 0.7 |
| HNPY 2-36 | 0.37 | 0.9 | 0.29 | 0.9 | 0.80 | 0.7 |
| HNPY 3-36 | 2.10 | 0.7 | 1.20 | 1.2 | 1.90 | 0.6 |
| HNPY 13-36 | 20.00 | 0.7 | 10.30 | 1.0 | 23.40 | 0.5 |
| HPP | 0.53 | 0.6 | 0.15 | 0.8 | 0.31 | 0.7 |
| D-Trp 32 NPY | 8.00 | 0.7 | 2.30 | 0.8 | 15.60 | 0.9 |

### Example 6

### Functional Assays of Chimeric NPY Receptors

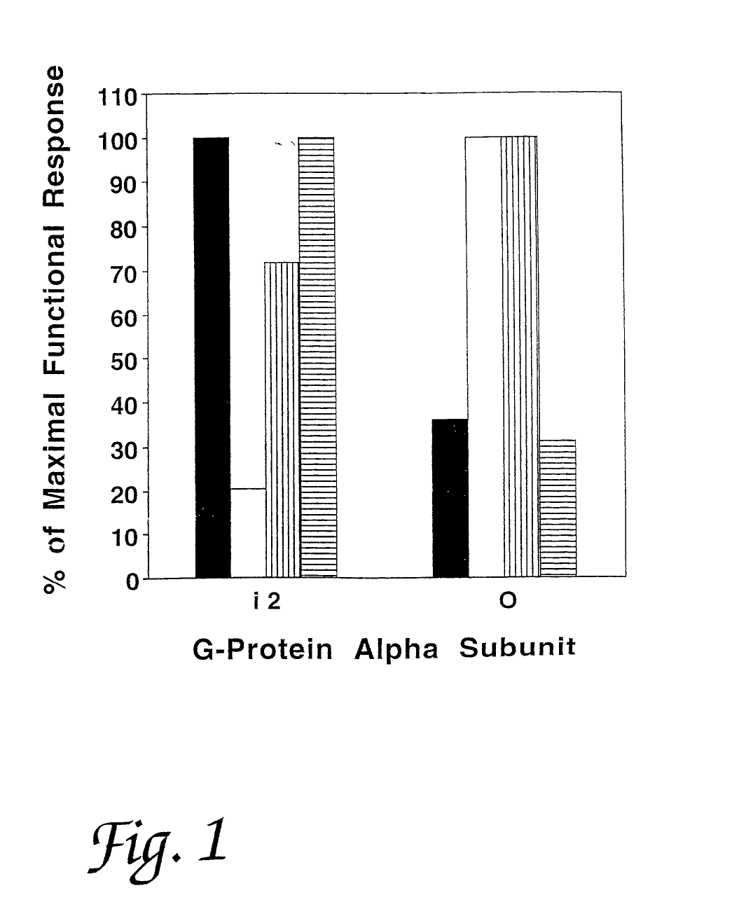

GTPγ³⁵S binding activity was measured using a modification of the method of Wieland and Jacobs, Methods Enzymol 237:3-13, 1994. Results are set forth in FIG. 1.

For each receptor construct tested, four baculoviral expression vector stocks were used to infect a culture of Sf9 cells (as described above in Example 3) with an MOI of 1:1:1:1. These four consisted of one vector encoding the NPY receptor construct being tested (prepared as described above) and a different commercially obtained baculoviral expression vector stock encoding each of the three subunits of a heterotrimeric G-protein.

In particular, the NPY expression vector constructs, as indicated in FIG. 1, were those comprising, in appropriate orientation for expression, the Y1 receptor cDNA of SEQ ID NO:1 (filled bars), the NPY5 receptor cDNA of SEQ ID NO:4 (open bars), the chimeric NPY5ΔY1CT receptor cDNA of SEQ ID NO:7 (vertical stripes), or the chimeric NPY5ΔY1IC3 receptor cDNA of SEQ ID NO:5 (horizontal stripes). The G-protein-encoding virus stocks were obtained from BIOSIGNAL Inc., Montreal, and were 1) a Gα G-protein subunit-encoding virus stock as indicated in FIG. 1 below the X axis (wherein i2 indicates the rat Gα₁₂ G-protein-encoding virus stock BIOSIGNAL #V5J008 and O indicates the rat Gαₒ G-protein-encoding virus stock BIOSIGNAL #V5H010), 2) a bovine β1 G-protein-encoding virus stock (BIOSIGNAL #V5H012), and 3) a human γ2 G-protein-encoding virus stock (BIOSIGNAL #V6B003). Agonist-stimulated GTPγ³⁵S binding on purified membranes was assessed using hNPY 1-36 (American Peptide Co., Sunnyvale, Calif.) as agonist in order to ascertain which receptor/Gαβγ combination(s) yielded the maximal functional activity as measured by GTPγ³⁵S binding.

Purified membranes, prepared by the method given above in Example 4, were resuspended by Dounce homogenization (tight pestle) in GTPγ³⁵S binding assay buffer (50 mM Tris pH 7.0, 120 mM NaCl, 2 mM MgCl2, 2 mM EGTA, 0.1% BSA, 0.1 mM bacitracin, 100KIU/mL Aprotinin, 5 μM GDP) and added to reaction tubes at a concentration of 30 μg/reaction tube. After adding increasing doses of the agonist hNPY 1-36 (American Peptide Co., Sunnyvale, Calif.), reactions were initiated by the addition of 100 pM GTPγ³⁵S. Following a 30-minute incubation at room temperature, the reactions were terminated by vacuum filtration over GF/C filters (pre-soaked in wash buffer, 0.1% BSA) followed by washing with ice-cold wash buffer (50 mM Tris pH 7.0, 120 mM NaCl).

Bound GTPγ³⁵S was determined by liquid scintillation spectrometry of the washed filters. Non-specific binding was determined using 10 mM GTPγS and represented less than 5 percent of total binding. Data is expressed as % maximal response and was derived by determining the maximal agonist stimulated % above basal stimulation for each receptor type, and normalizing all other data within that receptor type to the maximal (100%) value. The results of these GTPγ³⁵S binding experiments were analyzed using SIGMAPLOT software (SPSS Inc., Chicago).

Results are shown in FIG. 1 and discussed in the Brief Description of the Drawings.

The data suggest that G protein subtypes are functionally distinct, affecting receptor/Gαβγ binding interactions and consequently the maximal functional activity of the native and chimeric receptors as measured by the GTP for GDP exchange on the G alpha submit of the G-protein complex.

1. A chimeric receptor protein comprising a single polypeptide chain of amino acids, said protein comprising, in N-terminal to C-terminal order and immediately adjacent to each other and without further intervening amino acids, the following amino acid sequence domains:
a) an NPY5 receptor N-terminal extracellular domain,
b) an NPY5 receptor first transmembrane domain,
c) an NPY5 receptor first intracellular loop domain,
d) an NPY5 receptor second transmembrane domain,
e) an NPY5 receptor first extracellular loop domain,
f) an NPY5 receptor third transmembrane domain,
g) an NPY5 receptor second intracellular loop domain,
h) an NPY5 receptor fourth transmembrane domain,
i) an NPY5 receptor second extracellular loop domain,
j) an NPY receptor fifth transmembrane domain,
k) an NPY1 receptor third intracellular loop domain,
l) an NPY receptor sixth transmembrane domain,
m) an NPY5 receptor third extracellular loop domain,
n) an NPY5 receptor seventh transmembrane domain, and
o) an NPY5 receptor C-terminal intracellular domain.

2. A chimeric receptor protein according to claim 1, in which the NPY receptor of the fifth transmembrane domain and the sixth transmembrane domain are selected from NPY1 and NPY5 receptors.

3. A chimeric receptor protein according to claim 1, in which each domain is independently selected from human, monkey, dog, mouse, pig, guinea pig, and rat receptors.

4. A chimeric receptor protein comprising a single polypeptide chain of amino acids, said protein comprising, in N-terminal to C-terminal order and immediately adjacent to each other and without further intervening amino acids, the following amino acid sequence domains:
a) an NPY5 receptor N-terminal extracellular domain,
b) an NPY5 receptor first transmembrane domain,
c) an NPY5 receptor first intracellular loop domain,
d) an NPY5 receptor second transmembrane domain,
e) an NPY5 receptor first extracellular loop domain,
f) an NPY5 receptor third transmembrane domain,
g) an NPY5 receptor second intracellular loop domain,
h) an NPY5 receptor fourth transmembrane domain,
i) an NPY5 receptor second extracellular loop domain,
j) an NPY5 receptor fifth transmembrane domain,
k) an NPY5 receptor third intracellular loop domain,
l) an NPY5 receptor sixth transmembrane domain,
m) an NPY5 receptor third extracellular loop domain,
n) an NPY receptor seventh transmembrane domain, and
o) an NPY1 receptor C-terminal intracellular domain.

5. A chimeric receptor protein according to claim 4, in which the NPY receptor of the fifth transmembrane domain and the sixth transmembrane domain are selected from NPY1 and NPY5 receptors.

6. A chimeric receptor protein according to claim 4, in which each domain is independently selected from human, monkey, dog, mouse, pig, guinea pig, and rat receptors.

7. A chimeric receptor protein comprising a single polypeptide chain of amino acids, said protein comprising, in N-terminal to C-terminal order and immediately adjacent to each other and without further intervening amino acids, the following amino acid sequence domains:
a) an NPY5 receptor N-terminal extracellular domain,
b) an NPY5 receptor first transmembrane domain,
c) an NPY5 receptor first intracellular loop domain,
d) an NPY5 receptor second transmembrane domain,
e) an NPY5 receptor first extracellular loop domain,
f) an NPY5 receptor third transmembrane domain,
g) an NPY5 receptor second intracellular loop domain,
h) an NPY5 receptor fourth transmembrane domain,
i) an NPY5 receptor second extracellular loop domain,
j) an NPY receptor fifth transmembrane domain,
k) an NPY 1 receptor third intracellular loop domain,
l) an NPY receptor sixth transmembrane domain,
m) an NPY5 receptor third extracellular loop domain,
n) an NPY receptor seventh transmembrane domain, and
o) an NPY1 receptor C-terminal intracellular domain.

8. A chimeric receptor protein according to claim 7, in which the NPY receptor of the fifth transmembrane domain and the sixth transmembrane domain are selected from NPY1 and NPY5 receptors.

9. A chimeric receptor protein according to claim 7, in which each domain is independently selected from human, monkey, dog, mouse, pig, guinea pig, and rat receptors.

10. An isolated polynucleotide encoding a polypeptide comprising the chimeric receptor protein of claim 1, the receptor protein consisting of the amino acid sequence of SEQ. ID NO. 6, or a fragment of said sequence capable of binding a signal transducing ligand for said receptor protein.

11. An isolated polynucleotide encoding a polypeptide comprising the chimeric receptor protein of claim 4, the receptor protein consisting of the amino acid sequence of SEQ. ID NO. 9, or a fragment of said sequence capable of binding a signal transducing ligand for said receptor protein.

12. An isolated polynucleotide encoding a polypeptide comprising the chimeric receptor protein of claim 7, the receptor protein consisting of the amino acid sequence of SEQ. ID NO. 10, or a fragment of said sequence capable of binding a signal transducing ligand for said receptor protein.

13. A nucleic acid molecule encoding the protein of claim 1.

14. A nucleic acid molecule encoding the protein of claim 4.

15. A nucleic acid molecule encoding the protein of claim 7.

16. An isolated polynucleotide encoding a chimeric receptor protein according to claim 1, the polynucleotide consisting of SEQ. ID. NO. 5 and homologues thereof or a polynucleotide which hybridizes to the complement of SEQ. ID. NO. 5.

17. An isolated polynucleotide encoding a chimeric receptor protein according to claim 4, the polynucleotide consisting of SEQ. ID. NO. 7 and homologues thereof or a polynucleotide which hybridizes to the complement of SEQ. ID. NO. 7.

18. An isolated polynucleotide encoding a chimeric receptor protein according to claim 7, the polynucleotide consisting of SEQ. ID. NO. 8 and homologues thereof or a polynucleotide which hybridizes to the complement of SEQ. ID. NO. 8.

19. A vector for recombinant expression of a chimeric receptor protein, said vector comprising the nucleic acid molecule of claim 13, operatively linked to at least one regulatory element in the appropriate orientation for expression.

20. A vector for recombinant expression of a chimeric receptor protein, said vector comprising the nucleic acid molecule of claim 14, operatively linked to at least one regulatory element in the appropriate orientation for expression.

21. A vector for recombinant expression of a chimeric receptor protein, said vector comprising the nucleic acid molecule of claim 15, operatively linked to at least one regulatory element in the appropriate orientation for expression.

22. A vector for recombinant expression of a chimeric receptor protein, said vector comprising the polynucleotide of claim 16, operatively linked to at least one regulatory element in the appropriate orientation for expression.

23. A vector for recombinant expression of a chimeric receptor protein, said vector comprising the polynucleotide of claim 17, operatively linked to at least one regulatory element in the appropriate orientation for expression.

24. A vector for recombinant expression of a chimeric receptor protein, said vector comprising the polynucleotide of claim 18, operatively linked to at least one regulatory element in the appropriate orientation for expression.

25. The vector of claim 19, wherein the vector is a plasmid vector.

26. The vector of claim 20, wherein the vector is a plasmid vector.

27. The vector of claim 21, wherein the vector is a plasmid vector.

28. The vector of claim 22, wherein the vector is a plasmid vector.

29. The vector of claim 23, wherein the vector is a plasmid vector.

30. The vector of claim 24, wherein the vector is a plasmid vector.

31. The vector of claim 19, wherein the vector is a viral vector.

32. The vector of claim 20, wherein the vector is a viral vector.

33. The vector of claim 21, wherein the vector is a viral vector.

34. The vector of claim 22, wherein the vector is a viral vector.

35. The vector of claim 23, wherein the vector is a viral vector.

36. The vector of claim 24, wherein the vector is a viral vector.

37. A recombinant cell comprising the vector of claim 19, said recombinant cell being prepared by introducing said vector into a host cell not containing said vector to generate a vector-containing cell containing said vector, wherein the recombinant cell is the vector-containing cell or its progeny.

38. A recombinant cell comprising the vector of claim 20, said recombinant cell being prepared by introducing said vector into a host cell not containing said vector to generate a vector-containing cell containing said vector, wherein the recombinant cell is the vector-containing cell or its progeny.

39. A recombinant cell comprising the vector of claim 21, said recombinant cell being prepared by introducing said vector into a host cell not containing said vector to generate a vector-containing cell containing said vector, wherein the recombinant cell is the vector-containing cell or its progeny.

40. A recombinant cell comprising the vector of claim 22, said recombinant cell being prepared by introducing said vector into a host cell not containing said vector to generate a vector-containing cell containing said vector, wherein the recombinant cell is the vector-containing cell or its progeny.

41. A recombinant cell comprising the vector of claim 23, said recombinant cell being prepared by introducing said vector into a host cell not containing said vector to generate a vector-containing cell containing said vector, wherein the recombinant cell is the vector-containing cell or its progeny.

42. A recombinant cell comprising the vector of claim 24, said recombinant cell being prepared by introducing said vector into a host cell not containing said vector to generate a vector-containing cell containing said vector, wherein the recombinant cell is the vector-containing cell or its progeny.

43. The recombinant cell of claim 37, wherein the recombinant cell exhibits neuropeptide Y binding activity that is at least 2-fold greater than that exhibited by the host cell.

44. The recombinant cell of claim 38, wherein the recombinant cell exhibits neuropeptide Y binding activity that is at least 2-fold greater than that exhibited by the host cell.

45. The recombinant cell of claim 39, wherein the recombinant cell exhibits neuropeptide Y binding activity that is at least 2-fold greater than that exhibited by the host cell.

46. The recombinant cell of claim 40, wherein the recombinant cell exhibits neuropeptide Y binding activity that is at least 2-fold greater than that exhibited by the host cell.

47. The recombinant cell of claim 41, wherein the recombinant cell exhibits neuropeptide Y binding activity that is at least 2-fold greater than that exhibited by the host cell.

48. The recombinant cell of claim 42, wherein the recombinant cell exhibits neuropeptide Y binding activity that is at least 2-fold greater than that exhibited by the host cell.

49. The recombinant cell of claim 43, wherein the host cell is an insect cell.

50. The recombinant cell of claim 44, wherein the host cell is an insect cell.

51. The recombinant cell of claim 45, wherein the host cell is an insect cell.

52. The recombinant cell of claim 46, wherein the host cell is an insect cell.

53. The recombinant cell of claim 47, wherein the host cell is an insect cell.

54. The recombinant cell of claim 48, wherein the host cell is an insect cell.

55. The recombinant cell of claim 43, wherein the host cell is a mammalian cell.

56. The recombinant cell of claim 44, wherein the host cell is a mammalian cell.

57. The recombinant cell of claim 45, wherein the host cell is a mammalian cell.

58. The recombinant cell of claim 46, wherein the host cell is a mammalian cell.

59. The recombinant cell of claim 47, wherein the host cell is a mammalian cell.

60. The recombinant cell of claim 48, wherein the host cell is a mammalian cell.

61. An amphibian oocyte comp rising an RNA which is the nucleic acid molecule of claim 13.

62. An amphibian oocyte comprising an RNA which is the nucleic acid molecule of claim 14.

63. An amphibian oocyte comprising an RNA which is the nucleic acid molecule of claim 15.

64. An amphibian oocyte comprising an RNA which is the polynucleotide of claim 16.

65. An amphibian oocyte comprising an RNA which is the polynucleotide of claim 17.

66. An amphibian oocyte comprising an RNA which is the polynucleotide of claim 18.

67. A preparation of recombinant membranes isolated from a plurality of the recombinant cell of claim 43, wherein the recombinant membranes of the preparation exhibit neuropeptide Y binding activity that is at least 2-fold greater than that exhibited by a control consisting of a matched preparation of membranes isolated from host cells.

68. A preparation of recombinant membranes isolated from a plurality of the recombinant cell of claim 44, wherein the recombinant membranes of the preparation exhibit neuropeptide Y binding activity that is at least 2-fold greater than that exhibited by a control consisting of a matched preparation of membranes isolated from host cells.

69. A preparation of recombinant membranes isolated from a plurality of the recombinant cell of claim 45, wherein the recombinant membranes of the preparation exhibit neuropeptide Y binding activity that is at least 2-fold greater than that exhibited by a control consisting of a matched preparation of membranes isolated from host cells.

70. A preparation of recombinant membranes isolated from a plurality of the recombinant cell of claim 46, wherein the recombinant membranes of the preparation exhibit neuropeptide Y binding activity that is at least 2-fold greater than that exhibited by a control consisting of a matched preparation of membranes isolated from host cells.

71. A preparation of recombinant membranes isolated from a plurality of the recombinant cell of claim 47, wherein the recombinant membranes of the preparation exhibit neuropeptide Y binding activity that is at least 2-fold greater than that exhibited by a control consisting of a matched preparation of membranes isolated from host cells.

72. A preparation of recombinant membranes isolated from a plurality of the recombinant cell of claim 48, wherein the recombinant membranes of the preparation exhibit neuropeptide Y binding activity that is at least 2-fold greater than that exhibited by a control consisting of a matched preparation of membranes isolated from host cells.

73. An assay for characterizing a test compound, said assay comprising contacting a chimeric receptor of claim 1, with the test compound and detecting a consequence of the binding of said test compound to said receptor.

74. An assay for characterizing a test compound, said assay comprising contacting a chimeric receptor of claim 4, with the test compound and detecting a consequence of the binding of said test compound to said receptor.

75. An assay for characterizing a test compound, said assay comprising contacting a chimeric receptor of claim 7, with the test compound and detecting a consequence of the binding of said test compound to said receptor.

76. The assay of claim 73, wherein the test compound is unlabeled and the consequence is the displacement from the receptor of a labeled compound that binds specifically to the receptor.

77. The assay of claim 74, wherein the test compound is unlabeled and the consequence is the displacement from the receptor of a labeled compound that binds specifically to the receptor.

78. The assay of claim 75, wherein the test compound is unlabeled and the consequence is the displacement from the receptor of a labeled compound that binds specifically to the receptor.

79. The assay of claim 73, wherein the receptor is a membrane-inserted receptor and the consequence is a response associated with at least one intracellular domain of the receptor.

80. The assay of claim 74, wherein the receptor is a membrane-inserted receptor and the consequence is a response associated with at least one intracellular domain of the receptor.

81. The assay of claim 75, wherein the receptor is a membrane-inserted receptor and the consequence is a response associated with at least one intracellular domain of the receptor.

82. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 73.

83. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 74.

84. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 75.

85. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 76.

86. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 77.

87. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 78.

88. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 79.

89. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 80.

90. A method of treating a condition in a subject selected from eating disorders, seizure disorders, blood pressure disorders, locomoter disorders and anxiety disorders, which comprises administering to the subject a therapeutically effective amount of a composition comprising a compound identified as modulating the activity of an NPY receptor by carrying out the assay of claim 81.
